(19) United States
(12) Patent Application Publication
(10) Pub. No.: US 2016/0220313 A1
(43) Pub. Date: Aug. 4, 2016

(54) INSTRUMATION FOR THROACIC SURGERY

(71) Applicant: COVIDIEN LP, Mansfield, MA (US)

(72) Inventors: Ravi Durvasula, Irvine, CA (US); Haiying Liu, Winchester, MA (US)

(21) Appl. No.: 14/917,624

(22) PCT Filed: Aug. 12, 2014
(86) PCT No.: PCT/US2014/050641

§ 371 Date: Mar. 9, 2016

Related U.S. Application Data
(60) Provisional application No. 61/875,335, filed on Sep. 9, 2013.

(51) Int. Cl. A61B 34/20; A61B 5/00; A61B 1/00; A61B 1/313; A61B 17/00; A61B 1/05

(52) U.S. Cl. CPC A61B 34/20; A61B 17/00234; A61B 1/051; A61B 1/00045; A61B 1/00009; A61B 1/3132; A61B 5/0077; A61B 2017/00296; A61B 2034/2057

(57) ABSTRACT

A multi-modality imaging device is provided for use in surgical procedures. The disclosed imaging device incorporates a line of sight surface tissue imaging device and a sub-surface tissue imaging device into a single instrument to facilitate sub-surface surgical procedures. The line of sight surface tissue imaging device may be movably mounted relative to the sub-surface tissue imaging device and movable between a retained position and a deployed position offset from a longitudinal axis of the sub-surface tissue imaging device. Alternatively, the line of sight surface tissue imaging device may be stationary and concentric around the sub-surface tissue imaging device. There is also disclosed a system including a multi-modality imaging device mounted on a surgical instrument.

### CROSS-REFERENCE TO RELATED APPLICATIONS

This application is a U.S. National Stage Application filed under 35 U.S.C. §371(a) of International Patent Application No. PCT/US2014/050641, filed Aug. 12, 2014, which claims benefit of, and priority to, U.S. Provisional Patent Application Ser. No. 61/875,335, filed on Sep. 9, 2013, the entire content of which is incorporated herein by reference.

### BACKGROUND

1. Technical Field

The present disclosure relates to an imaging device for viewing tissues during surgery. More particularly, the present disclosure relates to an imaging device incorporating surface and sub-surface tissue imaging modalities for simultaneously viewing surface and sub-surface tissues in real time.

2. Background of Related Art

During many surgical or radiological procedures, it is very helpful for health care providers to visualize both surface level features as well as features embedded in tissue or sub-surface features. For example, there is often a need for surgeons to have the ability to identify the location and geometry of underlying nodules or tumors. This is particularly useful during video assisted surgeries such as, for example, video assisted thoracic surgery (VATS).

This technique typically requires multiple imaging devices which are used separately. Initially, a camera or other surface imaging device is used to identify an operative site. Then, the camera is removed and a sub-surface imaging probe is placed at the site and utilized to image the underlying tissue including the targeted nodules or tumors. This takes extra time and may result in positional inaccuracy due to the insertion and removal of multiple imaging devices within the operative site.

Therefore, there exists a need for a single imaging device having multiple imaging modalities including surface tissue and sub-surface tissue imaging. There further exists a need for an imaging device which can generate surface and sub-surface images simultaneously.

### SUMMARY

There is disclosed an imaging device for use in visualizing surface and sub-surface tissues during a surgical procedure. The imaging device generally includes a body portion, a line of sight surface imaging device connected to the body portion and a sub-surface imaging device connected to the body portion. The line of sight surface imaging device is offset from a longitudinal axis of the sub-surface imaging device and is movable between a retained position substantially in alignment with the sub-surface imaging device to a deployed position offset from a longitudinal axis of the sub-surface imaging device

The sub-surface imaging device may be coaxial with the body portion and extends from a distal end of the body portion. The sub-surface imaging device is a probe having a case and a sensor while the line of sight surface imaging device is a camera.

In one embodiment, the body portion includes a bay and the camera is movable between the deployed position offset from the body portion and the retained position wherein the camera resides in the bay.

In a specific embodiment, the camera is flexibly connected to the body portion. In an alternative specific embodiment, the camera is pivotally connected to the body portion.

In a further alternative embodiment, a case of the probe includes a bay and the camera is movable between the deployed position offset from the case of the probe and the retained position wherein the camera resides in the bay.

In a still further embodiment, the line of sight surface imaging device is coaxial with the sub-surface imaging device. In a more specific further embodiment, the line of sight surface imaging device is a ring of lenses that radially surrounds the sub-surface imaging device.

There is also disclosed a system for positioning and visualizing surface and sub-surface tissue. The system includes a surgical instrument having an elongate tubular member and an imaging device mounted on the elongate tubular member and including a line of sight surface imaging device and a sub-surface imaging device.

In one embodiment of the system, the imaging device is movably mounted on the elongate tubular member. In a more specific embodiment, the imaging device is offset from a longitudinal axis of the elongate tubular member.

The disclosed system further includes a display screen connected to the imaging device for viewing surface and sub-surface images generated by the line of sight surface imaging device and the sub-surface imaging device.

The system still further includes a converter for receiving the images generated and combining them into a merged image visible on the display screen.

There is also disclosed a method of positioning a sub-surface imaging probe on surface tissue and imaging sub-surface tissues. The method includes providing an imaging device having a body portion, a line of sight surface imaging device connected to the body portion and a sub-surface imaging device connected to the body portion. Surface tissue is viewed with the line of sight surface imaging device. The position of the sub-surface imaging device is viewed relative to the imaged surface tissue with the line of sight surface imaging device. The sub-surface imaging device is then positioned relative to the surface tissue and the sub-surface tissue is imaged with the now properly positioned sub-surface imaging device.

In a specific embodiment of the disclosed method, the images generated by the line of sight surface imaging device and sub-surface imaging device are viewed simultaneously in real time on a display screen.

### DESCRIPTION OF THE DRAWINGS

Various embodiments of the presently disclosed multi-modality imaging device are disclosed herein with reference to the drawings, wherein:

FIG. 1 is a perspective view of one embodiment of a multi-modality imaging device mounted on a surgical instrument;

FIG. 1A is an end view of the multi-modality imaging device and surgical instrument of FIG. 1;

FIG. 2 is a perspective view of the multi-modality imaging device with a line of sight imaging device in a retained condition;

FIG. 3 is a perspective view of the multi-modality imaging device with the line of sight imaging device in a deployed condition;

FIG. 4 is a perspective view, with parts separated, of the multi-modality imaging device;

FIG. 5 is a perspective view, with parts separated, of an alternative embodiment of a multi-modality imaging device;

FIG. 6 is a perspective view of the multi-modality imaging device of FIG. 5;

FIG. 7 is a perspective view of another alternative embodiment of a multi-modality imaging device;

FIG. 8 is a perspective view, with parts separated, of the multi-modality imaging device of FIG. 7;

FIG. 9 is a perspective view of a further alternate embodiment of a multi-modality imaging device with a line of sight imaging device in a retained condition;

FIG. 10 is a perspective view of the multi-modality imaging device of FIG. 9 with the line of sight imaging device in a deployed condition;

FIG. 11 is a perspective view of a still further alternate embodiment of a multi-modality imaging device;

FIG. 12 is an end view of the multi-modality imaging device of FIG. 11;

FIG. 13 is a plan view of an imaging system incorporating the multi-modality imaging device and surgical instrument of FIG. 1;

FIG. 14 is an image view of a sub-surface imaging probe of the multi-modality imaging device positioned against tissue taken from the line of sight device;

FIG. 15 is a three dimensional image of tissue taken by the sub-surface imaging probe; and

FIG. 16 is a compound image of FIGS. 14 and 15.

### DETAILED DESCRIPTION OF EMBODIMENTS

Embodiments of the presently disclosed multi-mode imaging device will now be described in detail with reference to the drawings wherein like numerals designate identical or corresponding elements in each of the several views. As is common in the art, the term ‘proximal” refers to that part or component closer to the user or operator, i.e. surgeon or physician, while the term “distal” refers to that part or component further away from the user.

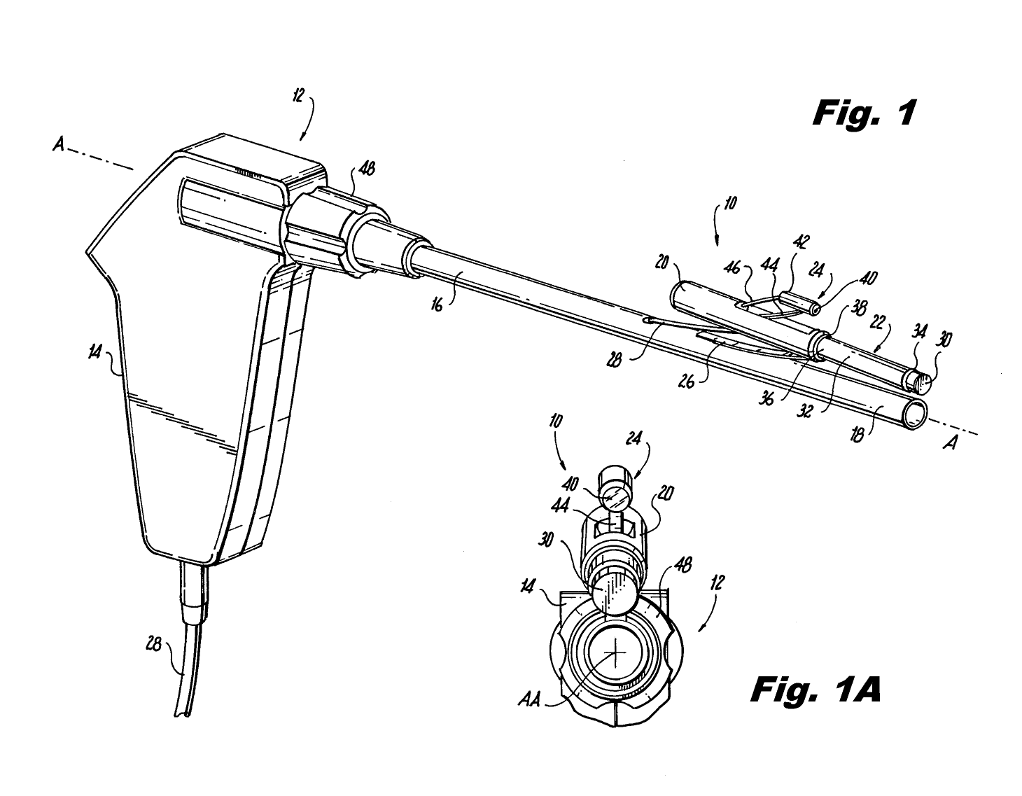

Referring initially to FIG. 1, there is disclosed one embodiment of a multi-mode imaging device or imaging device 10 for use in video assisted surgery along with a surgical instrument 12. Imaging device 10 can be used as a standalone instrument or can be mounted on surgical instrument 12. Surgical instrument 12 can be any of various types of surgical or radiological instrumentation. As shown, surgical instrument 12 generally includes a body portion 14 having an elongate tubular member 16 extending distally from body portion 14. Imaging device 10 is mounted on surgical instrument 12 proximal of a distal end 18 of elongate tubular member 16.

Imaging device 10 is provided to produce surface and sub-surface tissue images and generally includes a body portion 20, a sub-surface tissue imaging device or probe 22 and a line of sight surface tissue imaging device or camera 24. As used herein, the term “probe” 22 refers to sub-surface imaging devices including, but not limited to, ultrasound, photo-acoustic and/or fluorescent imaging devices while the term “camera” 24 refers to surface imaging devices including, but not limited to, visible cameras, near or mid-infrared cameras, fiber optic bundles, etc. As shown, imaging device 10 is movably mounted to elongate tubular member 16 of surgical instrument 12 by a flexible arm or stem 26. A cable 28 extends from body portion 20 of imaging device 10 and through surgical instrument 12 to relay images provided by probe 22 and camera 24 to a remote video screen (not shown).

Probe 22 includes a lens or sensor 30 and a body portion or case 32. Sensor 30 is provided for generating sub-tissue surface images and is mounted on a distal end 34 of a case 32. In this embodiment, a proximal end 36 of case 32 extends from a distal end 38 of body portion 20.

Camera 24 includes a lens 40 and a case 42. In this embodiment, camera 42 is movably mounted to body portion 20 by a flexible arm or stem 44. Camera 24 is movable between a retained position within body portion 20 and a deployed position (shown) offset from body portion 20 and probe 22. A cable 46 extends from camera 24 and is in communication with cable 28 to transmit images to a remote display.

Referring to FIGS. 1 and 1A, imaging device 10 is offset from a longitudinal axis A-A of elongate tubular member 16 of surgical instrument 12. Camera 24 is also offset from probe 22. This allows imaging device 10 to simultaneously view both surface and sub-surface tissue being operated on, or viewed by, surgical instrument 12. In order to better position imaging device 10 relative to an operative site, surgical instrument 12 includes a rotation knob 48 affixed to elongate tubular member 16 and rotatably mounted on body portion 14 of surgical instrument 12.

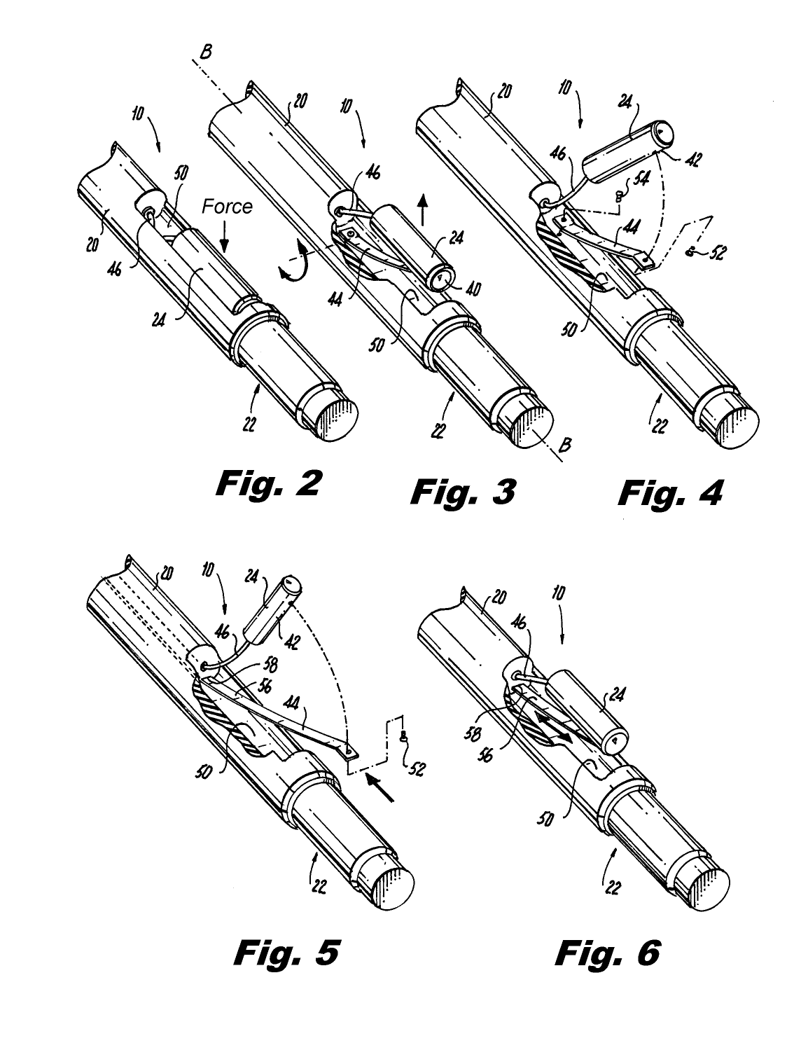

Referring now to FIGS. 2-4, in this embodiment, camera 24 is at least partially retained within a bay 50 formed in body portion 20 of imaging device 10. Camera 24 is movable from a retained position at least partially within bay 50 of body portion 20 (FIG. 2) to a deployed position off set from a longitudinal axis B-B of body portion 20 and probe 22 (FIG. 3). This allows camera 24 to “see around” probe 22 and facilitate the placement of probe 22 against tissue. Various methods can be used to “release” camera 24 from bay 50 and return camera 24 to within bay 50, such as, for example, a sliding cover, etc.

With reference to FIGS. 3 and 4, camera 24 is movably mounted to body portion 20 by flexible stem 44. Flexible stem 44 may be formed integrally with body portion 20 or formed as a separate member out of a flexible material such as, for example, spring steel, etc. As best shown in FIG. 4, when formed as a separate member, flexible stem 44 is affixed to case 42 of camera 24 by a fastener 52 and to body portion 20 by a fastener 54.

Referring to FIGS. 5 and 6, in an alternative embodiment, a proximal end 56 of flexible stem 44 extends through a slot formed in body portion 20 of imaging device 10 and is movable there through. Case 42 of camera 24 is still affixed to flexible stem 44 by fastener 52. By exerting a distal force and a proximal tension on proximal end 56 of flexible stem 44, camera 24 can be extended out of and withdrawn into bay 50 of body portion 20 of imaging device 10 to move camera 24 between the retained and deployed positions.

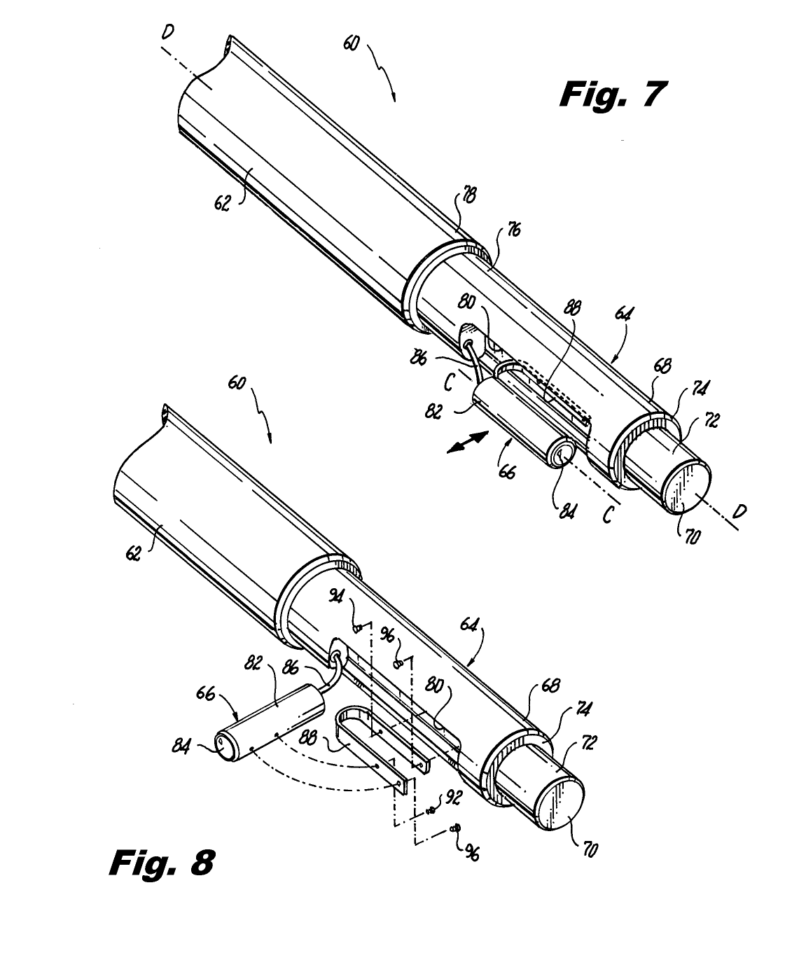

Referring now to FIGS. 7 and 8, there is disclosed an alternate embodiment of a multi-mode imaging device 60. Imaging device 60 generally includes a body portion 62, a sub-surface tissue probe 64 and a camera 66. Probe 64 includes a case 68 extending distally from body portion 62 and a sensor 70 extending distally from case 68. Specifically, a proximal end 72 of sensor 70 extends from a distal end 74 of case 68. A proximal end 76 of case 68 extends from a distal end 78 of body portion 62. In this embodiment, camera 66 is movably mounted to probe 64 and is retained at least partially within a bay 80 formed in case 68 of probe 64. Camera 66 is movable from a retained position contained at least partially within bay 80 to a deployed position out of bay 80 such that a longitudinal axis C-C of camera 66 is offset from a longitudinal axis D-D case 68 of probe 64 and body portion 62. This, again, allows camera 66 to view around probe 64 to visualize the operative site and assist in positioning probe 64 against tissue.

Camera 66 includes a case 82 having a lens 84 for viewing an operative site and assisting in positioning probe 64 against tissue. A cable 86 extends from case 82 of camera 66 to a display (not shown). In order to move camera 66 from the retained position to the deployed position, a U-spring 88 is provided between camera 66 and case 82. Fasteners 90 and 92 secure U-spring 88 to camera 66 while fasteners 94 and 96 secure U-spring 88 to case 82 and within bay 80.

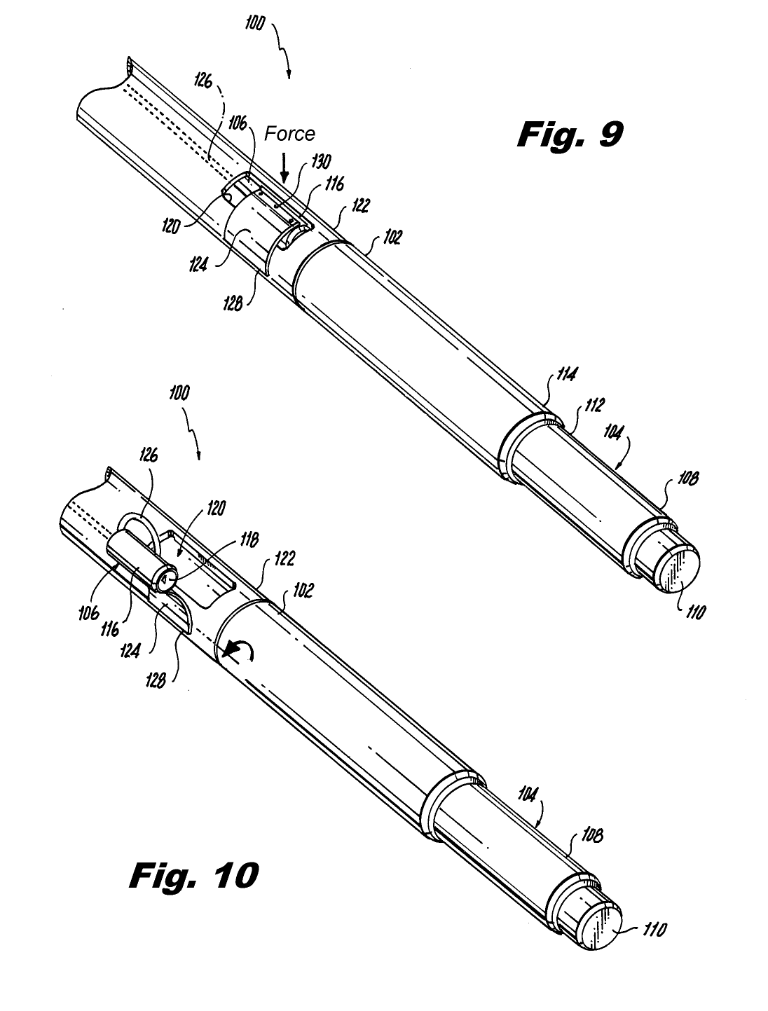

Referring now to FIGS. 9 and 10, there is disclosed a further alternate embodiment of an imaging device 100. Similar to prior embodiments, imaging device 100 generally includes a body portion 102, a sub-surface imaging probe 104 and an extendable, surface viewing camera 106. Probe 104 includes a case 108 and a sensor 110. A proximal end 112 of case 108 extends distally from a distal end 114 of body portion 102. Camera 106 generally includes a case 116 and a lens 118.

Similar to imaging device 10 described hereinabove, camera 106 is retained at least partially within a bay 120 formed in body portion 102. In this particular embodiment, body portion 102 includes a proximally extending body extension 122 defining bay 120. A hinge 124 is provided to move camera 106 from the retained position at least partially within bay 120 to a deployed position out of bay 120 and offset from body portion 102 and probe 104. A cable 126 extends from camera 106 through body portion 102 to a display device (not shown).

Hinge 124 can be formed as a living hinge integral with body portion 102 or as a separate pivoting member. In this embodiment, and in contrast to prior embodiments, camera 106 is pivoted out of bay 120 and away from body portion 102 and probe 104 rather than being biased away laterally by a spring or linkage. Specifically, hinge 124 is connected to body portion extension by a pivot 128 and to camera 106 by fasteners 130.

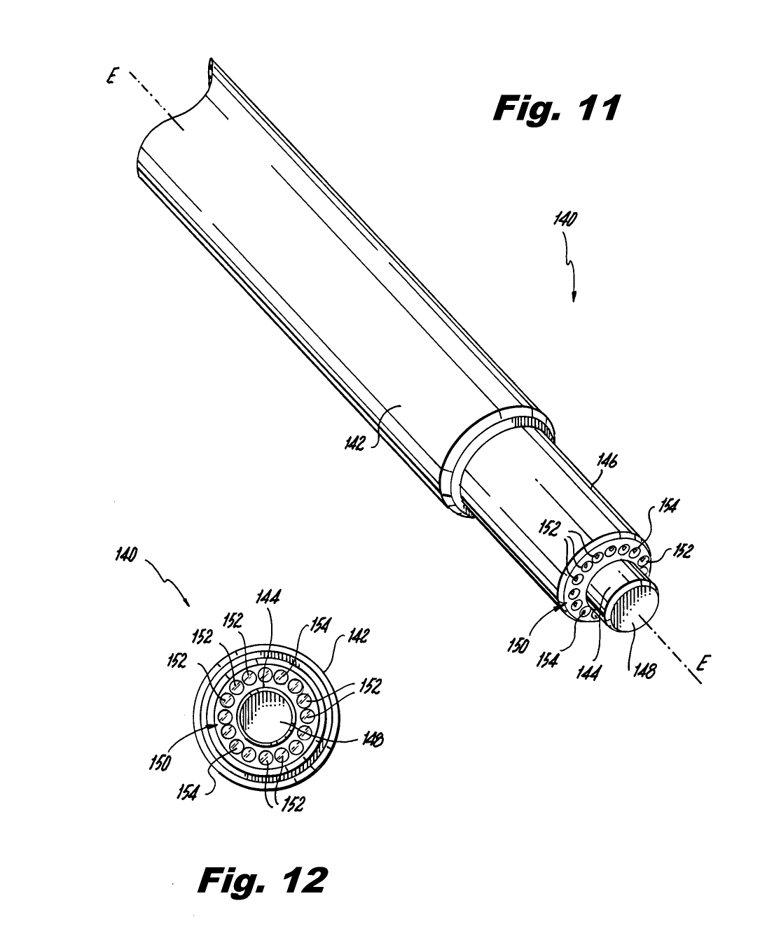

Turing now to FIGS. 11 and 12, there is disclosed yet a further alternate embodiment of an imaging device 140. Imaging device 140 includes a body portion 142, a sub-surface probe 144 and a camera 146. Sub-surface probe 144 is coaxial with body portion 142 about longitudinal axis E-E and includes a sensor 148.

In this embodiment, camera 146 is also coaxial with body portion 142, as well as sub-surface probe 144. Camera 146 includes a ring of lenses 150 positioned circumferentially around sensor 148 of probe 144. Individual lenses 152 of ring of lenses 150 may be utilized individually to image an area alongside of probe 144 or may be utilized simultaneously to generate a circular image around probe 144 to assist in positioning probe 144 precisely against surface tissue. Further, circle of lenses 150 may incorporate one or more visible light sources 154 in place of an individual lens 152 to illuminate the surface tissue.

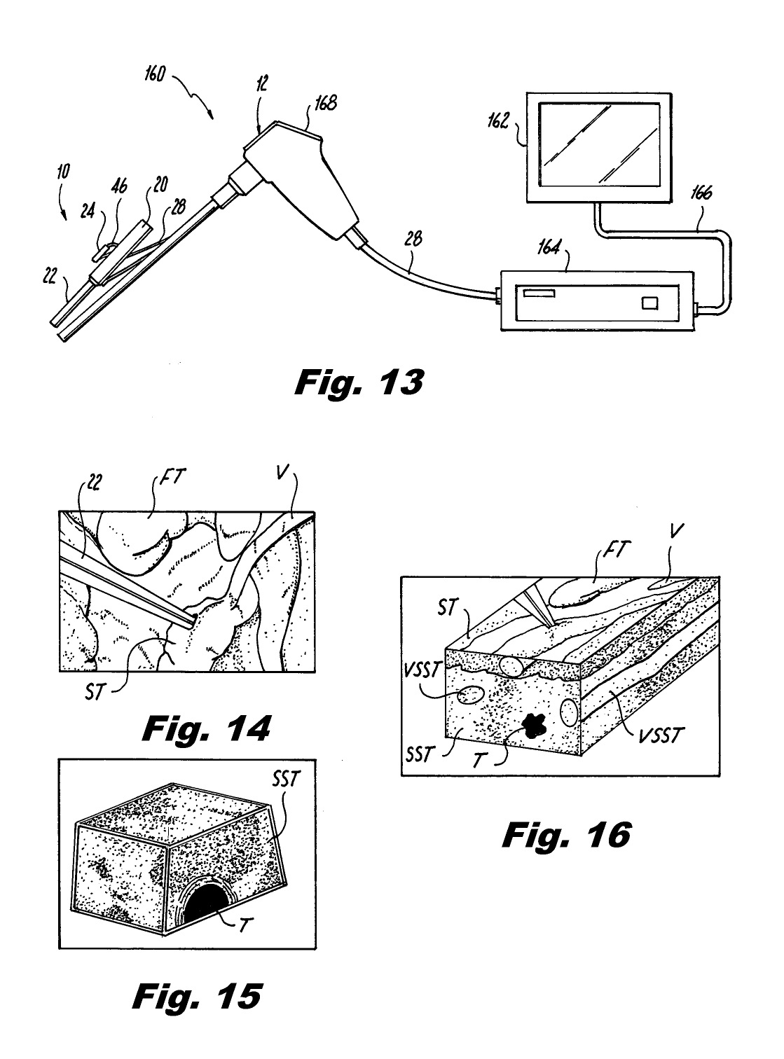

Turning now to FIGS. 13-15, there is disclosed an imaging system 160 and a method of use. Imaging system 160 generally includes surgical instrument 12 and a multi-mode imaging device, such as, for example, imaging device 10 described herein above. Imaging system 160 additionally includes a display screen 162 for viewing images generated by probe 22 and camera 24. A converter box 164 is provided to receive surface and sub-surface tissue image inputs though cable 28, process the image inputs and transmit them to display screen through cable 166. It should be noted that, while the disclosed system uses external display screen 162 and converter box 164, surgical instrument 12 may be equipped with a self contained converter and display screen 168 to operate as a single integrated unit.

Referring to FIGS. 13 and 14, probe 22 is positioned against surface tissue ST by visualizing its location with the aid of camera 24. By using camera 24 to view surface tissue ST, probe 22 can easily be positioned on surface tissue ST while avoiding other tissue structures such as, for example, veins V, fatty tissues FT, etc.

Referring to FIG. 15, once probe 22 has been properly positioned on surface tissue ST, a sub-surface image of sub-surface tissue structures SST, such as tumor T, can be generated and viewed on screen 162. The images of the surface tissue ST (FIG. 14) generated by camera 24 and the sub-surface tissue SST generated by probe 22 may be viewed on display screen 162 as side-by-side images on a split screen (not shown) or may be manipulated by converter box 164 to generate a single, real time, three dimensional image of surface tissue ST and underlying sub-surface tissues SST and tumor T (FIG. 15).

The disclosed images can then be used to perform an operation on tumor T while avoiding surface tissue structures such as fatty tissue FT and veins V as well as avoiding any additional underlying tissues structures such as, for example, sub-surface veins VSST's, etc. In this manner imaging device 10 and system 160 provided a means of visualizing an operative area in real time, three dimensions.

It will be understood that various modifications may be made to the embodiments disclosed herein. For example, the disclosed line of sight surface imaging device may be movably mounted on other structure such as, for example, linkages, telescoping shafts, etc. Further, the disclosed lenses can be fixed angle, variable angle, wide angle, etc. Additionally, more than one probe or camera may be incorporated into the disclosed imaging devices. Therefore, the above description should not be construed as limiting, but merely as exemplifications of particular embodiments. Those skilled in the art will envision other modifications within the scope and spirit of the claims appended hereto.

1. An imaging device for use in visualizing tissue during a surgical procedure comprising:
a body portion;
a line of sight surface imaging device connected to the body portion; and
a sub-surface imaging device connected to the body portion.

2. The imaging device as recited in claim 1, wherein the line of sight surface imaging device is offset from a longitudinal axis of the sub-surface imaging device.

3. The imaging device as recited in claim 1, wherein the line of sight surface imaging device is movable between a retained position substantially in alignment with the sub-surface imaging device to a deployed position offset from a longitudinal axis of the sub-surface imaging device.

4. The imaging device as recited in claim 3, wherein the sub-surface imaging device is coaxial with the body portion.

5. The imaging device as recited in claim 4, wherein the sub-surface imaging device extends from a distal end of the body portion.

6. The imaging device as recited in claim 5, wherein the sub-surface imaging device is a probe having a case and a sensor.

7. The imaging device as recited in claim 6, wherein the line of sight surface imaging device is a camera.

8. The imaging device as recited in claim 7, wherein the body portion includes a bay and the camera is movable between the deployed position offset from the body portion and the retained position wherein the camera resides at least partially in the bay.

9. The imaging device as recited in claim 8, wherein the camera is flexibly connected to the body portion.

10. The imaging device as recited in claim 8, wherein the camera is pivotally connected to the body portion.

11. The imaging device as recited in claim 7, wherein the case of the probe includes a bay and the camera is movable between the deployed position offset from the case of the probe and the retained position wherein the camera resides at least partially in the bay.

12. The imaging device as recited in claim 1, wherein the line of sight surface imaging device is coaxial with the sub-surface imaging device.

13. The imaging device as recited in claim 12, wherein the line of sight surface imaging device is a ring that radially surrounds the sub-surface imaging device.

14. A system for positioning and visualizing surface and sub-surface tissue comprising:
a surgical instrument having an elongate tubular member; and
an imaging device mounted on the elongate tubular member and including a line of sight surface imaging device and a sub-surface imaging device.

15. The system as recited in claim 14, wherein the imaging device is movably mounted on the elongate tubular member.

16. The system as recited in claim 15, wherein the imaging device is offset from a longitudinal axis of the elongate tubular member.

17. The system as recited in claim 16, further comprising a display screen connected to the imaging device for viewing surface and sub-surface images generated by the line of sight surface imaging device and the sub-surface imaging device.

18. The system as recited in claim 17, further comprising a converter for receiving the images generated and combining them into a merged image visible on the display screen.

19. A method of imaging sub-surface tissue comprising:
providing an imaging device having a body portion, a line of sight surface imaging device connected to the body portion and a sub-surface imaging device connected to the body portion;
imaging surface tissue with the line of sight surface imaging device;
viewing a position of the sub-surface imaging device relative to the imaged surface tissue with the line of sight surface imaging device;
positioning the sub-surface imaging device relative to the surface tissue; and
imaging sub-surface tissue with the positioned sub-surface imaging device.

20. The method as recited in claim 19, wherein the images generated by the line of sight surface imaging device and sub-surface imaging device are viewed simultaneously in real time.
